(19) United States
(12) Patent Application Publication
(10) Pub. No.: US 2023/0157591 A1
(43) Pub. Date: May 25, 2023

(54) NON-INVASIVE DETECTION DEVICE FOR URIC ACID

(71) Applicant: TAIWAN REDEYE BIOMEDICAL INC., Hsinchu City (TW)

(72) Inventors: TSUNG-JUI LIN, HSINCHU CITY (TW); YU-HSUN CHEN, HSINCHU CITY (TW); SHUO-TING YAN, HSINCHU CITY (TW)

(21) Appl. No.: 17/530,886

(22) Filed: Nov. 19, 2021

(51) Int. Cl. A61B 5/145; A61B 5/20; A61B 5/00

(52) U.S. Cl. CPC A61B 5/14546; A61B 5/20; A61B 5/0059

(57) ABSTRACT

A non-invasive detection device for uric acid includes a waterproof casing, a monitor, a detection part, and a processor. The waterproof casing includes an internal space and a detection end. The monitor is mounted on the waterproof casing. The detection part includes a detection passage, a light source module, and at least one sensor. The detection passage is configured for urine to pass through. The light source module emits a detection beam with a first wavelength to the detection passage. The at least one sensor is mounted in the internal space, receives the detection beam penetrating the urine, and generates a light intensity signal according to the detection beam. The processor electrically connects to the monitor, the light source module, and the at least one sensor. The processor receives the light intensity signal, and calculates the uric acid content in the urine to generate a detection result.

### BACKGROUND OF THE INVENTION

### 1. Field of the Invention

The present invention relates to a detection device, more particularly a non-invasive detection device for uric acid.

### 2. Description of the Related Art

Uric acid is the final product after the body metabolizes nucleic acid (purine) of cells. Purine in the body is first metabolized into uric acid by the liver, then uric acid is sent to the kidneys through the blood circulation, and the kidneys eventually excrete the uric acid with urine. When the body produces too much uric acid or the kidneys have poor ability to metabolize uric acid, it is easy to cause symptoms such as hyperuricemia, gouty arthritis, nephrolithiasis, articular malformation, etc. Therefore, most people need to take blood or urine tests during health examination. Patients with hyperuricemia also need to check the uric acid content in blood and urine frequently to keep tracking their physical condition and reduce the chance of symptoms.

Currently, a uric acid test can be tested by blood, urine or test paper. However, the subjects need to fast for 8 to 10 hours before the blood test, and the blood test is an invasive test method, which is likely to cause discomfort for people who are prone to fainting or anemia. On the other hand, blood test and urine test both use optical detection and optical analysis, so the subject needs to go to a professional medical institution for testing. The existing blood test and urine test take several days for analysis, and the test results cannot be displayed to the subjects immediately, making the subjects unable to understand the immediate uric acid situation in the body. If the uric acid test paper is improperly maintained, it will be easily deteriorated by moisture in the air, affecting the accuracy of the uric acid test paper. Also, the resulting color indicator of the test paper must be interpreted by human eyes. Depending on the interpretation, different people can see different results. This means the result may involve human bias and lack precise measurements.

The current testing methods for uric acid are cumbersome and lack immediacy. Therefore, the current testing methods for uric acid need further improvements.

### SUMMARY OF THE INVENTION

The present invention provides a non-invasive detection device for uric acid. The detection device for uric acid detects the uric acid content in urine immediately, improves the detection efficiency, and improves the convenience of uric acid detection.

In order to achieve the foregoing purpose, the non-invasive detection device for uric acid includes a waterproof casing, a monitor, a detection part, and a processor.

The waterproof casing includes an internal space and a detection end.

The monitor is mounted on the waterproof casing.

The detection part is mounted on the detection end, and includes a detection passage, a light source module, and at least one sensor. The detection passage provides urine to pass. The light source module is mounted in the internal space, and emits a detection beam with a first wavelength to the detection passage. The at least one sensor is mounted in the internal space, receives the detection beam penetrating the urine, and generates a light intensity signal according to the detection beam.

The processor is mounted in the internal space and electrically connects to the monitor, the light source module, and the at least one sensor. The processor receives the light intensity signal, and calculates the uric acid content in the urine to generate a detection result according to the light intensity signal.

Wherein, the processor outputs the detection result to the monitor and displays the detection result in a display screen of the monitor.

The present invention emits the detection beam with the first wavelength by the light source module. The processor generates the light intensity signal by receiving the detection beam penetrating the urine. The processor calculates the uric acid content in the urine according to the light intensity signal and displays the detection result on the monitor for the subjects to know their physical condition in time. Compared to the conventional uric acid testing methods, the subjects of the present invention can perform uric acid test anytime and anywhere according to their own needs, and get the detection result quickly, which effectively improves the detection efficiency of uric acid test. Furthermore, the subjects do not need to go to a professional medical institution for uric acid test. Therefore, the present invention further improves the convenience of uric acid detection, and is convenient for the subject to perform uric acid detection in an instant, convenient and efficient manner to understand their own physical condition.

### BRIEF DESCRIPTION OF THE DRAWINGS

FIG. 1 is a perspective view of a first embodiment of a non-invasive detection device for uric acid.

FIG. 2 is a sectional view of a detection part of the non-invasive detection device for uric acid in the first embodiment.

FIG. 3 is a light path schematic diagram of the detection part of the non-invasive detection device for uric acid in the first embodiment.

FIG. 4 is a sectional view of the detection part of the non-invasive detection device for uric acid in a second embodiment.

FIG. 5 is a light path schematic diagram of the detection part of the non-invasive detection device for uric acid in the second embodiment.

FIG. 6 is a block diagram of the detection part of the non-invasive detection device of the present invention.

FIG. 7 is an absorbance spectrogram of a uric acid solution.

FIG. 8 is an absorbance spectrogram of various substances in urine.

FIG. 9 is a light intensity schematic diagram of the urine and uric acid solution for uric acid detection.

### DETAILED DESCRIPTION OF THE INVENTION

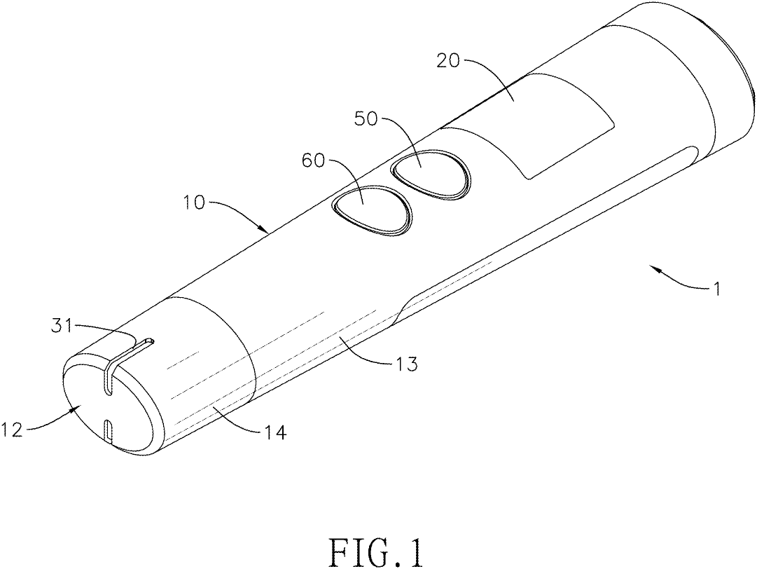

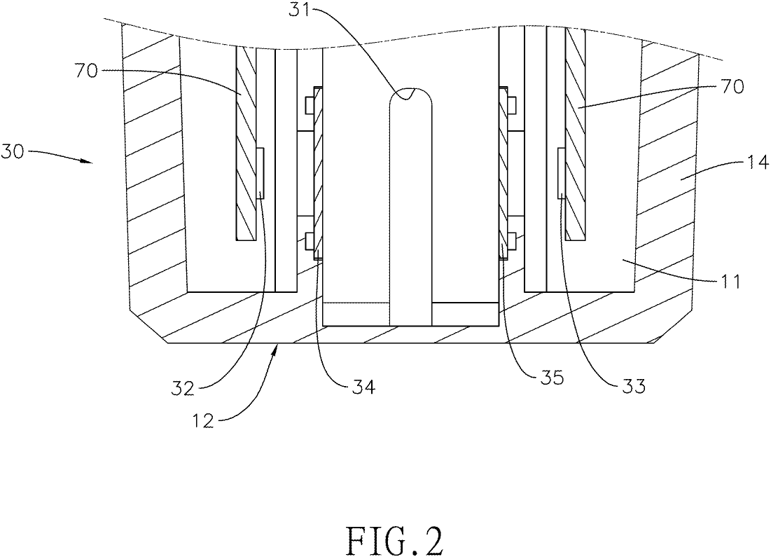

With reference to FIG. 1 and FIG. 2, in a first embodiment, the present invention of a non-invasive detection device for uric acid 1 includes a waterproof casing 10, a monitor 20, a detection part 30, and a processor 40. The waterproof casing 10 includes an internal space 11 and a detection end 12. When using the non-invasive detection device for uric acid 1, the detection end 12 must be set in a container containing urine to detect the uric acid content in the urine of the subjects. The waterproof casing 10 can isolate the internal space 11 from urine, prevent urine from penetrating into the internal space 11, and provide waterproof protection for the internal space 11.

The non-invasive detection device for uric acid 1 further includes a switch button 50 and a detection button 60. The switch button 50 and the detection button 60 are mounted on the waterproof casing 10 and electrically connected to at least one circuit board 70 in the internal space 11. The switch button 50 is used to turn on or off the non-invasive detection device for uric acid 1, and the detection button 60 is used to trigger the non-invasive detection device for uric acid 1 to perform uric acid detection.

The monitor 20 is mounted on the waterproof casing 10 and exposed outside the waterproof casing 10. The monitor 20 can display the detection result in a display screen for users to check out.

With reference to FIG. 1, the waterproof casing 10 further includes a first shell 13 and a second shell 14. The first shell 13 and the second shell 14 are connected to form the internal space 11. The monitor 20, the switch button 50 and the detection button 60 are mounted on the first shell 13, the detection end 12 is located at the second shell 14, and the detection part 30 is mounted on the second shell 14.

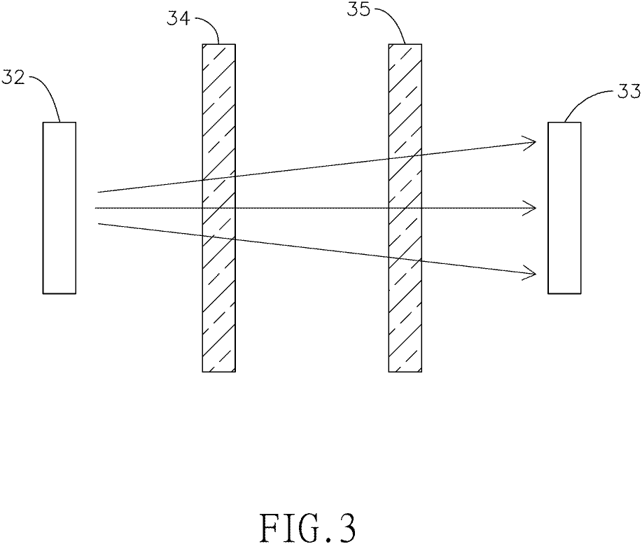

With reference to FIG. 3, the detection part 30 is mounted on the detection end 12 of the waterproof casing 10. The detection part 30 includes a detection passage 31, a light source module 32, and at least one sensor 33. In the current embodiment, the number of the at least one sensor 33 is one. The detection passage 31 is located at the detection end 12. When the detection part 30 is placed in a container containing urine, the detection passage 31 can allow urine to pass through, and a width of the detection passage 31 is 0.1 to 10 mm. The light source module 32 and the at least one sensor 33 are mounted on opposite sides of the detection passage 31, and mounted on the least one circuit board 70 in the internal space 11. A first lens 34 and a second lens 35 are respectively mounted on opposite sides of the detection passage 31. The first lens 34 is located between the light source module 32 and the detection passage 31. The second lens 35 is located between the detection passage 31 and the sensor 33. Wherein, the first lens 34 and the second lens 35 can be respectively isolated from urine by a waterproof cover. In other words, the waterproof cover is provided between the urine and the first lens 34, and the waterproof cover can also be provided between the urine and the second lens 35. The waterproof cover prevents urine from penetrating into the internal space 11, and the waterproof cover can be a transparent waterproof cover without affecting the functions of the first lens 34 and the second lens 35.

The light source module 32 emits a detection beam with a first wavelength to the detection passage. The detection beam sequentially penetrates the first lens 34, the urine in the detection passage 31, and the second lens 35, and is received by the sensor 33. The sensor 33 generates a light intensity signal according to the detection beam received. Wherein, the light source module 32 can be a light-emitting element such as a mercury lamp, a gas lamp, a laser light source, an LED lamp, etc. The sensor 33 can be a photosensitive element such as a light sensor, a photodiode array (PDA) sensor, a spectrometer, a complementary metal oxide semiconductor (CMOS) sensor, etc. The light intensity signal is expressed in terms of light absorbance. In this embodiment, the number of the at least one sensor 33 can also be plural, so as to increase the receiving area of the detection beam, and improve the accuracy of the light intensity signal.

According to the formula of Beer-Lambert law A = aLc and A = - log

$$- \text{log} \frac{I}{{I}_{\text{o}}} ,$$

10, A is the light absorbance, α is the molar attenuation coefficient, L is the optical path length, c is the concentration, I₀is the incident light intensity, I is the transmitted light intensity. When light enters an object, the light-absorbing material in the object will absorb part of the light energy, so that the light intensity of the light transmitted out of the object will be weakened. The absorbed energy can be regarded as light absorbance A. Therefore, the light absorbance A of the object can be calculated from the energy difference between the incident light intensity I₀and the transmitted light intensity I. When the detection part 30 is placed in urine for uric acid detection, the detection beam emitted by the light source module 32 passes through the urine in the detection passage 31 and is received by the sensor 33. Part of the light energy of the detection beam is absorbed by the uric acid in the urine, so the sensor 33 can generate the light intensity signal from the light intensity of the detection beam, and the concentration of uric acid in the urine can be calculated by the light absorbance. Wherein, the light intensity signal is expressed in terms of light absorbance.

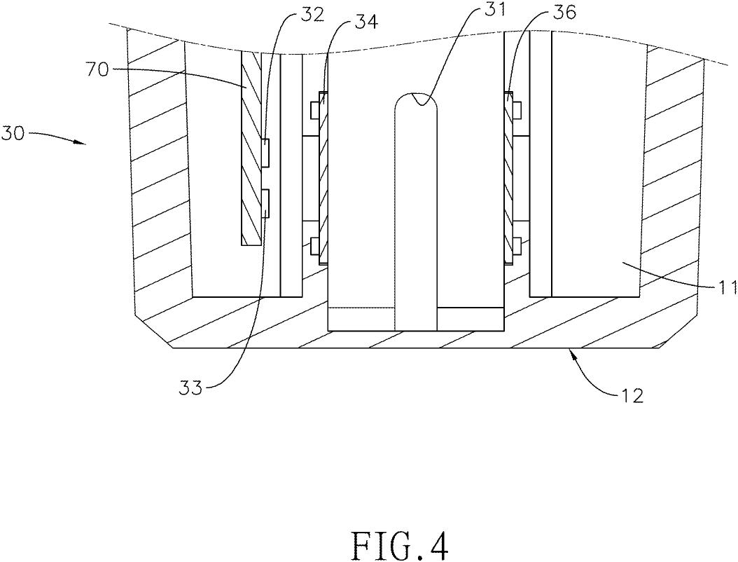

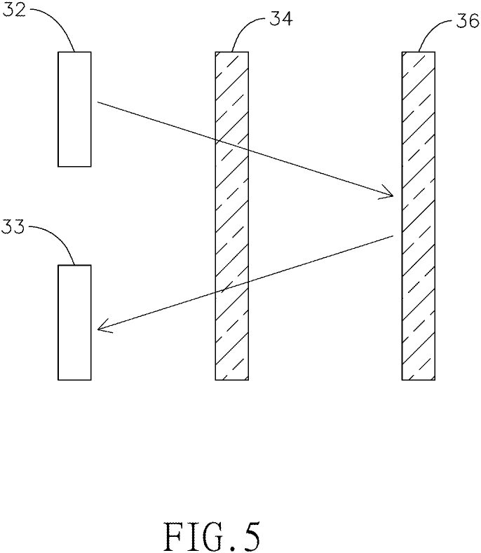

With reference to FIG. 4 and FIG. 5, in a second embodiment, a first lens 34 and a reflective lens 36 are respectively mounted on opposite sides of the detection passage 31. The light source module 32 and the sensor 33 are both mounted on the at least one circuit board 70 on the side of the detection passage 31 where the first lens 34 is mounted. When the light source module 32 emits a detection beam, the detection beam sequentially passes through the first lens 34 and the detection passage 31, and is reflected by the reflection lens 36. The detection beam is reflected by the reflection lens 36 received by the sensor 33 after passing through the detection passage 31 and the first lens 34. In this way, the path of the detection beam passing through the urine in the detection passage 31 will be extended, and the accuracy of the detection of uric acid will be further improved.

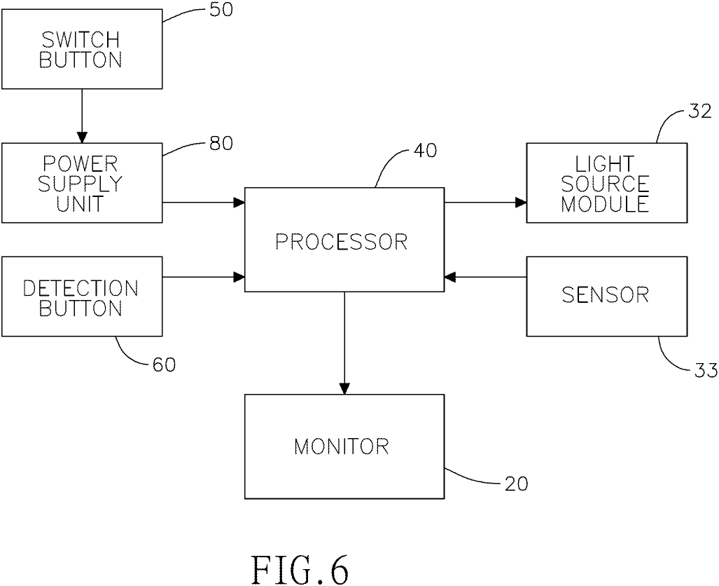

With reference to FIG. 6, the processor 40 is the operation control unit of the non-invasive detection device for uric acid 1. The processor 40 is mounted on the at least one circuit board 70 of the internal space 11, and the processor 40 can be a central processing unit or a microcontroller. The processor 40 is electrically connected to the monitor 20, the light source module 32, the at least one sensor 33, and the detection button 60. When the switch button 50 is triggered, the switch button 50 generates a starting signal to a power supply unit 80 of the non-invasive detection device for uric acid 1. The power supply unit 80 is mounted in the internal space 11 and electrically connected to the processor40 and the switch button 50. The power supply unit 80 can be a battery, and the power supply unit 80 supplies power to the processor 40 after receiving the starting signal. When the detection button 60 is triggered, the detection button 60 generates a detection signal to the processor 40. The processor 40 controls the light source module 32 to emit the detection beam according to the detection signal. When the detection beam penetrates the urine in the detection passage 31 and is received by the sensor 33, the sensor 33 generates the light intensity signal according to the light intensity of the received detection beam, and transmits the light intensity signal to the processor 40. The processor 40 calculates the uric acid content of the detected urine by the light intensity signal and generates a detection result. The processor 40 transmits the detection result to the monitor 20, and displays the detection result on the display screen of the monitor 20.

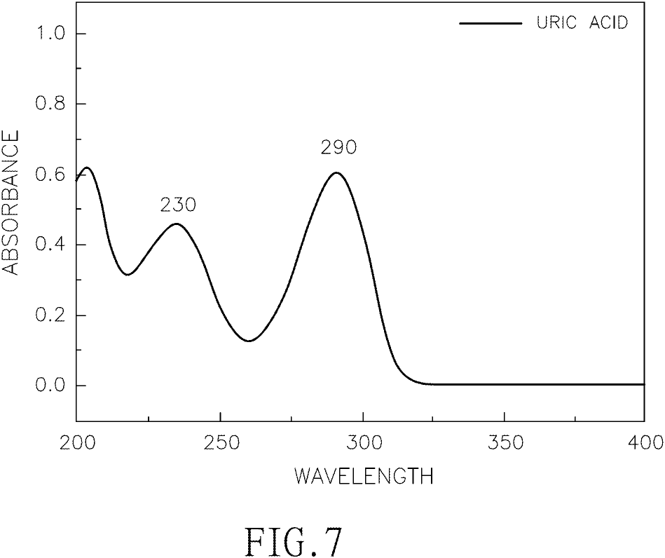

With reference to FIG. 7, the light absorbance peak of uric acid solution is in the wavelength range of 275 nm to 315 nm. In other words, if the detection beams of different wavelengths penetrate urine sample, the light energy of the detection beams with wavelengths from 275 nm to 315 nm is absorbed by the uric acid in the urine, which is particularly obvious compared to other wavelength ranges. In the non-invasive detection device for uric acid 1 of the present invention, the light source module 32 emits the detection beam with the first wavelength to perform uric acid detection. In this embodiment, the first wavelength is 275 nm to 315 nm.

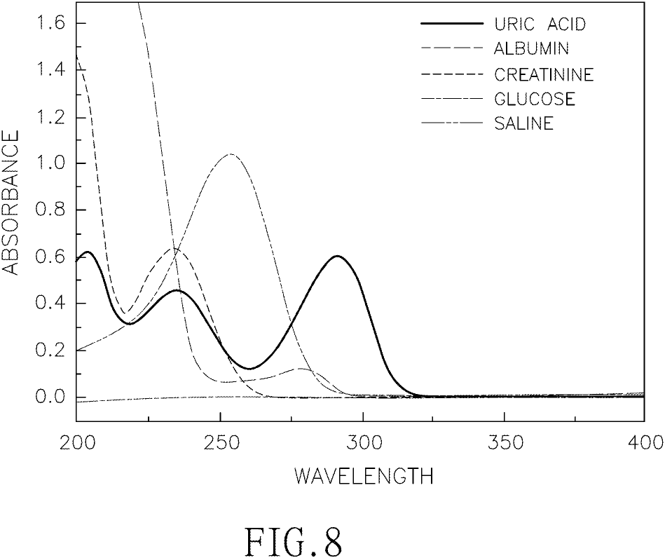

With reference to FIG. 8, it can be found from FIG. 8 that uric acid, albumin, creatinine, glucose, and salt in urine have different absorbance peaks, and the absorbance peak of uric acid does not overlap with the absorbance peak of other substances. In the wavelength range from 275 nm to 315 nm, the light absorbance of uric acid is particularly significant, while the light absorbance of other substances is not obvious. Therefore, if the detection beam with a wavelength of 275 nm to 315 nm is used for uric acid detection, it will not be interfered by other substances in the urine.

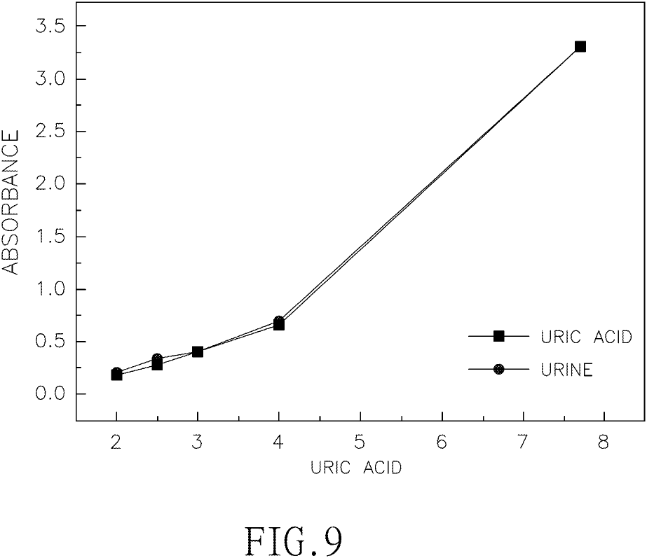

With reference to FIG. 9, the detection beam with a wavelength of 275 nm to 315 nm is used to detect uric acid in a uric acid solution and urine sample with the same uric acid concentration. When the uric acid concentration is 2 mg/dL, 2.5 mg/dL, 3 mg/dL, 4 mg/dL and 7.5 mg/dL, the light absorbance of the uric acid solution and the urine sample in the wavelength range from 275 nm to 315 nm is almost the same. It can be confirmed once again that only the light absorbance characteristics of uric acid are more significant in the wavelength range from 275 nm to 315 nm, and the concentration of other substances does not affect the accuracy of uric acid detection.

In addition, based on the relationship between the detection beam of different wavelengths and the concentration of uric acid, the processor 40 can use the light intensity signal to calculate a light absorbance intensity of uric acid in urine. The processor 40 calculates the uric acid content in urine by interpolation from the relationship between the light absorbance intensity transmitted through the detection beam and the uric acid concentration to generate the detection result.

On the other hand, when the non-invasive detection device for uric acid 1 detects uric acid in the diluted urine, the processor 40 can multiply the light absorbance learned from the light intensity signal by a dilution ratio of the urine after dilution, and calculate the actual uric acid content of the urine before the dilution to generate the detection result.

In summary, the non-invasive detection device for uric acid 1 of the present invention performs instant and rapid uric acid detection. When the detection part 30 has been placed in urine, the subject can press the detection button 60 to enable the processor 40. The processor 40 starts to control the light source module 32 to emit the detection beam with the first wavelength according to the detection signal. The sensor 33 receives the detection beam penetrating the urine to generate the light intensity signal. The processor 40 calculates the uric acid content in the urine according to the light intensity signal, and displays the detection result on the monitor 20, so that the subjects can know their physical condition in time. Compared with the conventional uric acid detection method, the present invention does not need to collect blood. The present invention performs uric acid detection through a non-invasive detection method, which can avoid the discomfort of the subject caused by blood collection. The invention does not need to use test paper or chemical reagents, which can effectively avoid the inaccurate detection caused by the deterioration of test paper or chemical reagents. In addition, the subject can perform uric acid detection through the present invention anytime and anywhere without the need to go to a professional medical institution, which can improve the convenience of uric acid detection and provide real-time health management.

What is claimed is:

1. A non-invasive detection device for uric acid, comprising:
a waterproof casing, including an internal space and a detection end;
a monitor, mounted on the waterproof casing;
a detection part, mounted on the detection end, including:
a detection passage, configured for urine to pass through;
a light source module, mounted in the internal space and emitting a detection beam with a first wavelength to the detection passage;
at least one sensor, mounted in the internal space, receiving the detection beam penetrating the urine, and generating a light intensity signal according to the detection beam;
a processor, mounted in the internal space and electrically connecting to the monitor, the light source module, and the at least one sensor, wherein the processor receives the light intensity signal, and calculates uric acid content in the urine to generate a detection result according to the light intensity signal;
wherein, the processor outputs the detection result to the monitor and displays the detection result in a display screen of the monitor.

2. The non-invasive detection device for uric acid as claimed in claim 1, wherein, the light source module and the at least one sensor are mounted on opposite sides of the detection passage.

3. The non-invasive detection device for uric acid as claimed in claim 1, comprising:
a first lens, located between the light source module and the detection passage;
a second lens, located between the at least one sensor and the detection passage;
wherein, the detection beam sequentially penetrates the first lens, the urine in the detection passage, and the second lens, and is received by the sensor.

4. The non-invasive detection device for uric acid as claimed in claim 1, comprising:
a first lens, located between the light source module and the detection passage;
a reflection lens, located between the light source module and the detection passage;
wherein, the light source module and the sensor are both mounted on a side of the detection passage where the first lens is mounted, and the detection beam sequentially passes through the first lens and the detection passage, and is reflected by the reflection lens;
wherein, the detection beam is reflected by the reflection lens received by the sensor after passing through the detection passage and the first lens.

5. The non-invasive detection device for uric acid as claimed in claim 1, wherein, the first wavelength is 275 nm to 315 nm.

6. The non-invasive detection device for uric acid as claimed in claim 1, comprising:
a power supply unit, mounted in the internal space and electrically connecting to the processor;
a switch button, mounted on the waterproof casing and electrically connecting to the power supply unit;
wherein, when the switch button is triggered, the switch button generates a starting signal to the power supply unit, and the power supply unit supplies power to the processor after receiving the starting signal.

7. The non-invasive detection device for uric acid as claimed in claim 1, comprising:
a detection button, mounted on the waterproof casing and electrically connecting to the processor;
wherein when the detection button is triggered, the detection button generates a detection signal to the processor, and the processor controls the light source module to emit the detection beam according to the detection signal.

8. The non-invasive detection device for uric acid as claimed in claim 1, wherein, a width of the detection passage is 0.1 to 10 mm.

9. The non-invasive detection device for uric acid as claimed in claim 1,
wherein, the waterproof casing further includes a first shell and a second shell;
the first shell and the second shell are connected to form the internal space; and
the monitor is mounted on the first shell, and the detection part is mounted on the second shell.

10. The non-invasive detection device for uric acid as claimed in claim 9, comprising:
a power supply unit, mounted in the internal space and electrically connecting to the processor;
a switch button, mounted on the waterproof casing and electrically connecting to the power supply unit, wherein, when the switch button is triggered, the switch button generates a starting signal to the power supply unit, and the power supply unit supplies power to the processor after receiving the starting signal; and
a detection button, mounted on the waterproof casing and electrically connecting to the processor, wherein when the detection button is triggered, the detection button generates a detection signal to the processor, and the processor controls the light source module to emit the detection beam according to the detection signal.
